(19) United States
(12) Patent Application Publication
(10) Pub. No.: US 2019/0218606 A1
(43) Pub. Date: Jul. 18, 2019

(54) METHODS OF REDUCING ERRORS IN DEEP SEQUENCING

(71) Applicant: Omigen (Hangzhou) Inc., Hangzhou (CN)

(72) Inventor: Yunguang TONG, Tucson, AZ (US)

(21) Appl. No.: 15/873,967

(22) Filed: Jan. 18, 2018

(51) Int. Cl. C12Q 1/6869; C12Q 1/6809

(52) U.S. Cl. CPC C12Q 1/6869; C12Q 2563/185; C12Q 1/6809

(57) ABSTRACT

A method to generate DMI (Digital Molecular Identifier) for sequencing read includes: obtaining a pool of signer nucleotides; mixing the signer nucleotides with target nucleotides; performing a reaction of adding the signer nucleotides to the target nucleotides to form signer-target nucleotides complexes; amplifying the signer-target nucleic acid complexes, resulting in a set of amplified signer-target nucleotides complexes; sequencing the amplified signer-target nucleotides complexes; and combining the information of the signer nucleotides, the target nucleotides, and the signer-target nucleotides complexes. A sequencing library preparation kit allowing computing DMI (Digital Molecular Identifier) includes a pool of nucleotides with known sequence serve as signer nucleotides; and reagents that allow the signer nucleotides to be randomly added to target nucleotides, thereby generating a molecular signature.

### FIELD OF THE INVENTION

The present invention relates to deep sequencing, more specifically a method of reducing errors in deep sequencing and a sequencing library preparation kit applying the method.

### BACKGROUND OF THE INVENTION

Deep sequencing has been widely used to investigate subpopulations in complex biological samples. Clinical applications, such as early detection of cancer and monitoring its response to therapy with nucleic acid-based serum biomarkers, have been developed. Tumor heterogenicity has been characterized through next-generation sequencing, and many low-frequency, drug-resistant variants of therapeutic importance have been identified.

There are still limitations of the sequencing technology because of errors introduced during sample preparation and sequencing. PCR amplification of heterogeneous mixtures can result in population skewing due to amplification biases and lead to over-representation or under-representation of particular variants. Polymerase mistakes during pre-amplification generate point mutations resulting from base mis-incorporations and rearrangements due to template switching. Combined with the additional errors that arise during cluster amplification, cycle sequencing and image analysis, approximately 1% of bases are incorrectly identified, depending on the specific platform and sequence context.

To overcome the limitations of the deep sequencing technology, a variety of improvements have been developed. For example, in a double strand SMI (single molecular identifier) method (WO 2013142389 A1, Methods of lowering the error rate of massively parallel DNA sequencing using duplex consensus sequencing), sequencing reads originating from each strand of DNA are recognized. The generated sequencing reads are then analyzed using double strand consensus sequences (DCSs) to remove errors. Although duplex sequencing theoretically can greatly reduce errors, it suffers from several drawbacks. First, the final SMI is a double strand, randomized sequence, which is hard to synthesize. The method shared by the same group (Nature Protocols 9, 2586-2606 (2014), Detecting ultralow-frequency mutations by Duplex Sequencing), used a single-stranded randomized sequence as a SMI template to obtain a double stand SMI and the quality control of double strand SMI adaptor requires radiolabeling and PAGE, which is challenging for a clinical lab. Second, due to the difficulty in making high quality SMI adaptors, the ligation efficiency might be compromised and therefore might requires large amount of input DNA. There is still a need to overcome the limitation of SMI based methods.

### SUMMARY OF THE INVENTION

An advantage of the present invention is to calculate DMI (Digital Molecular Identifier) using a pool of regular adapters with defined barcodes as signer nucleotides, which can be easily synthesized in high quality. Another advantage of the present invention is that different information of signer nucleotides and target nucleotides can be used for DMI calculation, based on the requirement of the study.

In one embodiment, a method to generate DMI (Digital Molecular Identifier) for sequencing read includes obtaining a pool of signer nucleotides; mixing the signer nucleotides with target nucleotides; performing a reaction of adding the signer nucleotides to the target nucleotides to form signer-target nucleotides complexes; amplifying the signer-target nucleic acid complexes, resulting in a set of amplified signer-target nucleotides complexes; sequencing the amplified signer-target nucleotides complexes; and combining the information of the signer nucleotides, the target nucleotides, and the signer-target nucleotides complexes.

In another embodiment, the signer nucleotides are adaptors with different molecular barcodes.

In another embodiment, the reaction is a ligation reaction allowing the signer nucleotides to be randomly ligated to the target nucleotides.

In another embodiment, the molecular barcodes are a single strand or double strand.

In another embodiment, the information includes sequence information, length of the target nucleotides and the location of the target nucleotides on a reference genome.

In one embodiment, a sequencing library preparation kit allowing computing DMI (Digital Molecular Identifier) includes a pool of nucleotides with known sequence serve as signer nucleotides; and reagents that allow the signer nucleotides to be randomly added to target nucleotides, thereby generating a molecular signature.

In another embodiment, the signer nucleotides are adaptors with a molecular barcode of at least 3 nucleotides in length.

In another embodiment, the target nucleotides are double-stranded DNA or RNA molecules.

In another embodiment, the signer nucleotides include at least two PCR primer binding sites, at least two sequencing primer binding sites, or both.

In another embodiment, the signer nucleotides are added to the target nucleotides via a ligation reaction.

In another embodiment, the signer nucleotides include a ligation adaptor selected from the group consisting of a T-overhang, an A-overhang, a CG overhang, a blunt end, and a ligatable nucleic acid sequence.

In another embodiment, the signer nucleotides are Y-shaped, U-shaped, or a combination thereof.

In another embodiment, the sequencing library preparation kit further includes a module to compute DMI by using the information of the signer nucleotides and target nucleotides. The information includes sequence information, the length of the target nucleotides and the location of the target nucleotides on a reference genome.

In one embodiment, a method of obtaining the sequence of a double-stranded target nucleic acid includes obtaining a pool of double-stranded signer nucleotides; mixing the double-stranded signer nucleotides with double-stranded target nucleotides; performing a reaction allowing the double-stranded signer nucleotides to be randomly added to double-stranded target nucleotides to form double-stranded signer-target nucleotides complexes; amplifying the double-stranded signer-target nucleotides complexes, resulting in a set of amplified signer-target nucleotides complexes; and sequencing the amplified double-stranded signer-target nucleotides complexes.

In another embodiment, the double-stranded target nucleotides are double-stranded DNA or RNA molecules.

In another embodiment, the method further includes generating an error-corrected single-stranded consensus sequence by (i) generating a DMI (Digital Molecular Identifier) using the information of the double-stranded signer-target nucleotides complexes; (ii) grouping the sequenced amplified signer-target nucleotides products into families of target nucleic acid strands based on the DMI; and (ii) removing target nucleic acid strands having one or more nucleotide positions where paired target nucleic acid strands disagree, or removing nucleotide positions from nucleic acid strands where single strands disagree at a specific position.

In another embodiment, the double-stranded target nucleotides are double-stranded circulating tumor DNA or reverse transcribed circulating tumor RNA fragment.

In another embodiment, the double-stranded nucleotides include a double-stranded target nucleic acid sequence ligation adaptor.

In another embodiment, the double-stranded target nucleic acid sequence ligation adaptor is selected from the group consisting of a T-overhang, an A-overhang, a CG overhang, a blunt end, and a ligatable nucleic acid sequence.

In another embodiment, each end of the double-stranded target nucleotides is ligated to a signer adaptor molecule.

In another embodiment, the signer adaptor molecule includes a molecular barcode sequence and an adaptor; the molecular barcode sequence includes a degenerate or semi-degenerate nucleic acid sequence; and the adaptor allows the signer adaptor molecule to be ligated to the double-stranded target nucleotides.

In another embodiment, the double-stranded signer nucleotide includes at least two PCR primer binding sites, at least two sequencing primer binding sites, a double-stranded fixed reference sequence, or a combination thereof.

In one embodiment, a method of generating an error corrected sequence includes obtaining a pool of signer nucleotides; mixing the pool of signer nucleotides with target nucleotides; performing a reaction allowing the signer nucleotides to be added to the target nucleotides to form signer-target nucleotides complexes; generating a set of PCR duplicates of the signer-target nucleotides complexes by performing PCR; sequencing the PCR duplicates; generating a DMI using the information of the signer-target nucleotides complexes; and creating a single strand consensus sequence using the DMI from the sequenced PCR duplicates which arose from an individual molecule of single-stranded DNA.

In another embodiment, the information includes the signer nucleotides and target nucleotides, the location of a target nucleotide on a reference genome, and the length of the target nucleotides.

In another embodiment, the method further includes comparing the sequence of two single strand consensus sequences arising from a single duplex DNA molecule; and reducing sequencing or PCR errors by (i) grouping the sequenced signer-target nucleic acid products into families of paired target nucleic acid strands based on a common set of DMI; and (ii) removing paired target nucleic acid strands having one or more nucleotide positions where paired target nucleic acid strands disagree, or removing nucleotide positions from nucleic acid strands where the paired strands disagree at a specific position.

In another embodiment, the signer nucleotides include a molecular barcode having at least 3 nucleotides in length.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 illustrates an overview of generating Digital Molecular Identifier (DMI). DMI takes three layers of information into consideration: the information of pooled signer nucleotides; the information of the target nucleotides and the randomness of forming signer-target complex. The DMI is calculated through using sequence information of the signer nucleotide and ligated target molecule. The sequencing reads are aligned to the genome to obtain location information of the target molecule in the genome.

FIG. 2 illustrates an overview of double strand error correction using DMI. Sheared double-stranded DNA molecules that have been end-repaired and T-tailed is combined with a pool of A-tailed molecular barcode adaptors and ligated in a randomized manner according to one embodiment. Every DNA fragment becomes labeled with two molecular barcode sequences. After size-selecting for appropriate length fragments, PCR amplification with primers containing Illumina flow-cell-compatible tails is carried out to generate families of PCR duplicates. Two types of PCR products are produced from each capture event. Those derived from one strand will have “A” molecular barcode sequence adjacent to flow-cell sequence 1 “a” and the “B” molecular barcode sequence adjacent to flow cell sequence 2 “b”. DMI is generated by combining AabB information. PCR products originating from the complementary strand are labeled reciprocally.

FIG. 3 illustrates an example of how to computing a DMI for DMI-based double strand error correction (DDSEC). Sequence of barcode on the signer nucleotide with n-mers of 3 nucleotides in length (3-mers) and sequencing of target nucleotide are read according to some embodiments. FIG. 3(A) shows the 3-mers barcode and 3-mers target nucleotide with the PCR primer binding sites (or flow cell sequences) 1 and 2 indicated at each end. FIG. 3(B) shows the same molecules as in (A) but with the strands separated and the lower strand now written in the 5′-3′ direction. When these molecules are amplified with PCR and sequenced, they will yield the following sequence reads: The top strand will give a read 1 file of TAA---CAT- and a read 2 file of GCC---TCG-. Combining the read 1 and read 2 tags will give TAACATCGGAGC as the DMI for the top strand. The bottom strand will give a read 1 file of CGG---AGC-- and a read 2 file of TAACAT-. Combining the read 1 and read 2 tags will give CGGAGTAACAT as the DMI for the bottom strand. FIG. 3(C) illustrates the orientation of paired strand mutations. In the initial DNA duplex shown in FIGS. 3A and 3B, a mutation “x” (which is paired to a complementary nucleotide “y”) is shown on the left side of the DNA duplex. The “x” will appear in read 1, and the complementary mutation on the opposite strand, “y,” will appear in read 2. Specifically, this would appear as “x” in both read 1 and read 2 data, because “y” in read 2 is read out as “x” by the sequencer owing to the nature of the sequencing primers, which generate the complementary sequence during read 2.

FIG. 4 illustrates error correction through DMI-based single strand error correction (DSSEC) and double strand error correction (DDSEC). According to one embodiment, (A-C) shows sequence reads sharing a unique set of molecular barcodes are grouped into paired families with members having strand identifiers in either the AabB or BbaA orientation. Each family pair reflects one double-stranded DNA fragment, (A) shows mutations (spots) present in only one or a few family members representing sequencing mistakes or PCR-introduced errors occurring late in amplification, (B) shows mutations occurring in many or all members of one family in a pair representing mutations scored on only one of the two strands, which can be due to PCR errors arising during the first round of amplification such as might occur when copying across sites of mutagenic DNA damage, (C) shows true mutations (* arrow) present on both strands of a captured fragment appear in all members of a family pair. While artifactual mutations may co-occur in a family pair with a true mutation, these can be independently identified and discounted when producing (D) an error-corrected consensus sequence (i.e., single stranded) for each duplex, (E) shows consensus sequences from all independently captured, randomly sheared fragments containing a particular genomic site are identified and (F) compared to determine the frequency of genetic variants at this locus within the sampled population.

FIG. 5 shows that DMI based consensus sequencing removes artifactual sequencing errors as compared to Raw Reads. DMI-based Double strand error correction (DDSEC) results in an approximately equal number of mutations as the reference and single strand error correction (DSSEC).

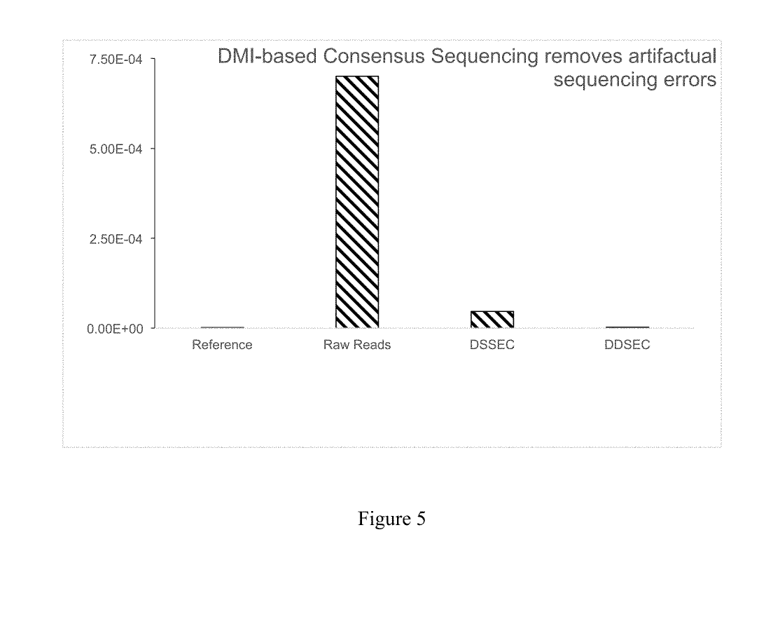

FIG. 6 shows DMI based duplex sequencing results in accurate recovery of spiked-control mutations. A series of variants of horizon sample, each harboring a known single-nucleotide substitution, were mixed in together at known ratios and the mixture was sequenced to 100,000-fold final depth. Standard sequencing analysis cannot accurately distinguish mutants present at a ratio of less than 1/100, because artifactural mutations occurring at every position obscure the presence of less abundant true mutations, rendering apparent recovery greater than 100%. DMI-based duplex consensus sequences, in contrast, accurately identify spiked-in mutations down to the lowest tested ratio of 1/50,000.

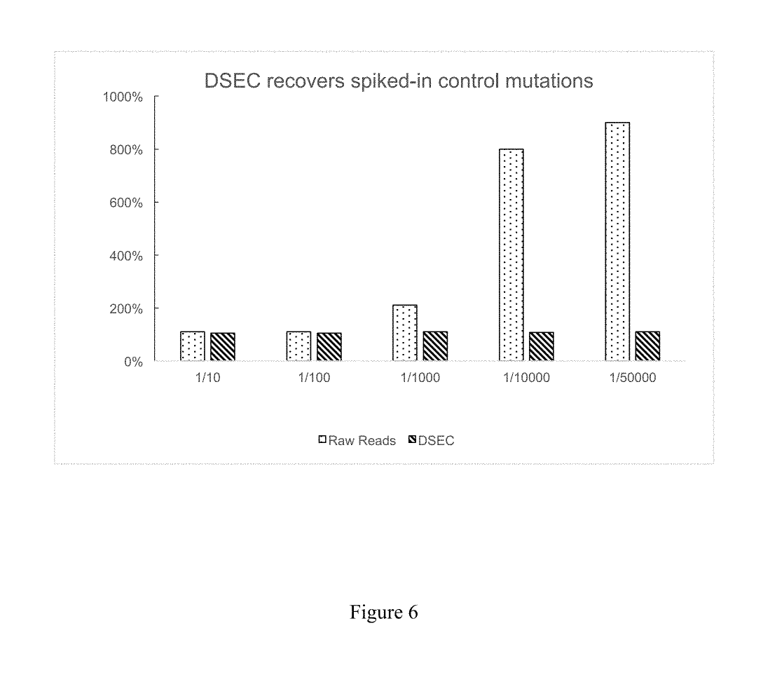

### DETAILED DESCRIPTION OF THE ILLUSTRATED EMBODIMENTS

Reference will now be made in detail to embodiments of the present invention, example of which is illustrated in the accompanying drawings.

### Definition:

Deep Sequencing refers to sequencing a genomic region multiple times, sometimes hundreds or even thousands of times.

Molecular Barcode is a unique n-mer sequence that is used to identify unique fragments and “de-duplicate” the sequencing reads from a sample.

Signer Nucleotide is a nucleotide with known sequences.

Molecular Signature refers to a molecular event, when a signer nucleotide joins a target molecular, a signer-target signature is generated.

Digital Molecular Identifier (DMI) refers to a set of parameters define the uniqueness of a signer-target nucleic acid complex.

DMI-based Single Strand Error Correction (DSSEC) is a method of removing sequencing errors by using a DMI for a single strand nucleotide.

DMI-based Double Strand Error Correction (DDSEC) is a method of removing sequencing errors by using a DMI set for a double strand nucleotide.

Provided herein is a new method, which employ DMI to identify PCR duplicates and remove errors through DMI-based single strand error correction (DSSEC) and double strand error correction (DDSEC). The advantage of DMI is its flexibility and allows for easy adjustment when needed.

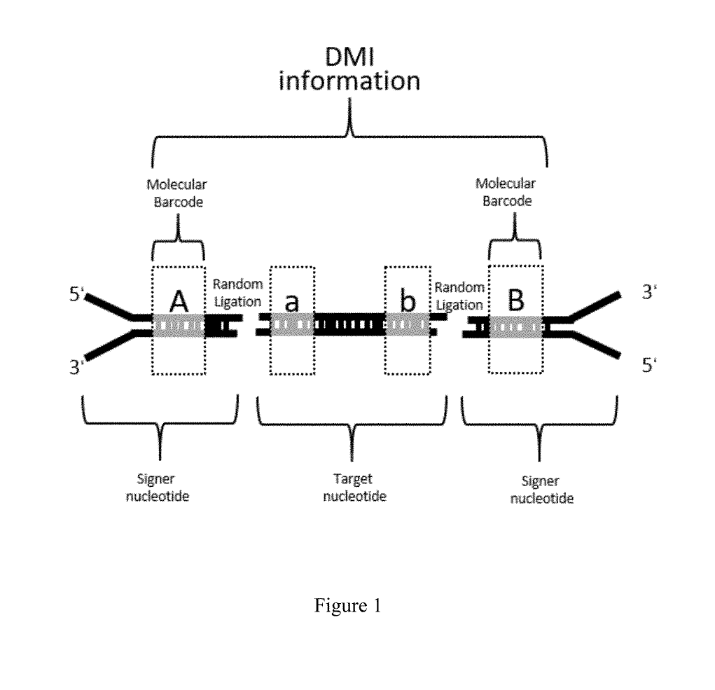

According to the embodiments described herein, a pool of signer nucleotides is provided. The signer nucleotides are mixed with target molecules and related reagents allowing target molecules to be ligated with the signer nucleotides. The signer nucleotides may be double stranded (FIG. 1) and include a molecular barcode. Optionally, each signer nucleotide further includes at least two PCR primer binding sites, at least two sequencing primer binding sites, or both. Signer-Target products are PCR amplified and sequenced. The target molecular sequence is obtained and mapped to a reference genome. The DMI is obtained by combining information of the signer nucleotides and target nucleotides in the singer-target nucleotide (nucleic acid) complexes. The used information of signer nucleotides and target nucleotides, includes but not limited to, sequence information, the location of the target molecular on the reference genome and length of target nucleotide and so on. DMI takes three layers of information into consideration: the information of pooled signer nucleotides, the information of the target nucleotides, and the randomness of the ligation join the signer nucleotide and target nucleotide.

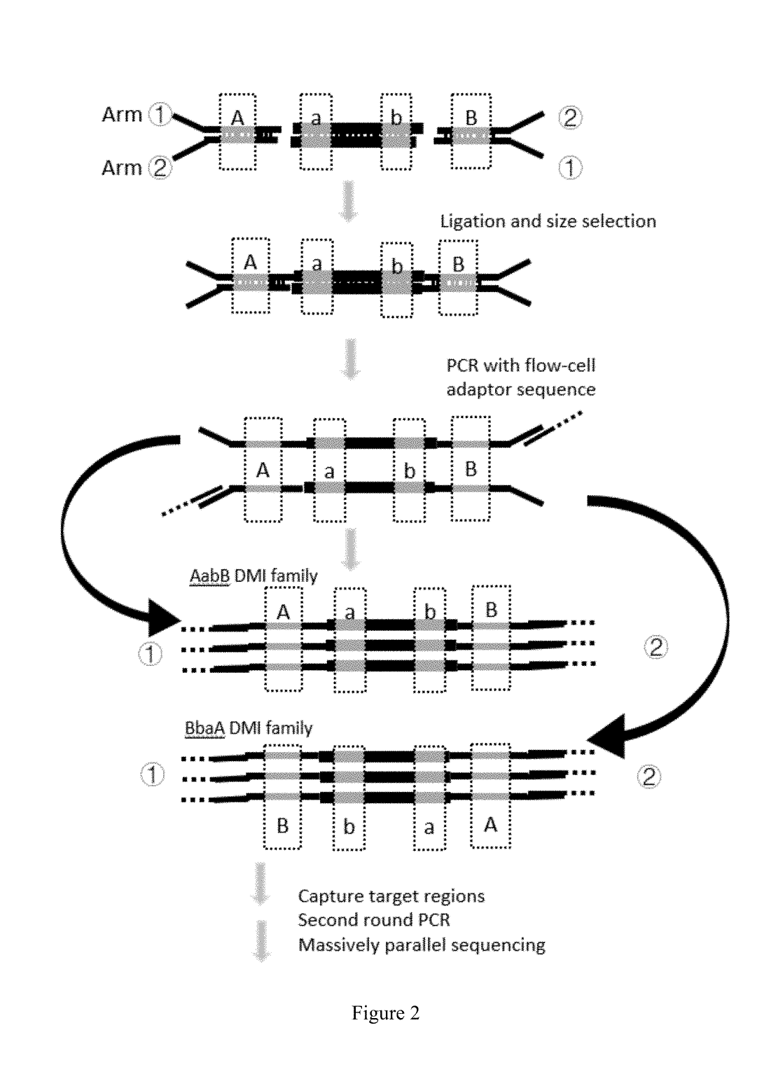

The signer nucleotides may be in the form of sequencing adaptor with molecular barcode, which may form a “Y-shape” or a “hairpin shape.” In some embodiments, the signer adaptor molecule is a “Y-shaped” adaptor, which allows both strands to be independently amplified by a PCR method prior to sequencing because both the top and bottom strands have binding sites for PCR primers FC1 and FC2 as shown in the examples below. A schematic of a Y-shaped signer adaptor molecule is also shown in FIG. 2. A Y-shaped signer adaptor requires successful amplification and recovery of both strands of the signer adaptor molecule. In one embodiment, a modification that would simplify consistent recovery of both strands entails ligation of a Y-shaped signer adaptor molecule to one end of a DNA duplex molecule, and ligation of a “U-shaped” linker to the other end of the molecule. PCR amplification of the hairpin-shaped product will then yield a linear fragment with flow cell sequences on either end. Distinct PCR primer binding sites (or flow cell sequences FC1 and FC2) will flank the DNA sequence corresponding to each of the two signer adaptor molecule strands, and a given sequence seen in Read 1 will then have the sequence corresponding to the complementary DNA duplex strand seen in Read 2. Mutations are scored only if they are seen on both ends of the molecule (corresponding to each strand of the original double-stranded fragment), i.e. at the same position in both Read 1 and Read 2. This design may be accomplished as described in the examples relating to double stranded DMI sequence tags.

In other embodiments, the signer adaptor molecule is a “hairpin” shaped (or “U-shaped”) adaptor. A hairpin DNA product can be used for error correction, as this product contains both of the two DNA strands. Such an approach allows for reduction of a given sequencing error rate N to a lower rate of N*N*(1/3), as independent sequencing errors would need to occur on both strands, and the same error among all three possible base substitutions would need to occur on both strands. For example, the error rate of 1/100 in the case of Illumina Sequencing would be reduced to (1/100)*(1/100)*(1/3)=1/30,000.

According to the embodiments described herein, the molecule barcode sequence (or “tag”) may be a double-stranded, complementary molecule barcode sequence. The molecule sequence is a fixed nucleotide n-mer sequence which is 12 nucleotides in length. For example, a pool (96) of nucleotide molecular barcode 12-mer sequence that is randomly ligated to each end of a target nucleic acid molecule results in generation of up to 9,216 distinct tag sequences.

In some embodiments, the molecular barcodes in the signer adaptor can be two single stranded sequences in a defined relationship, without being complementary or reserve complementary.

In some embodiments, the signer adaptor molecules are ligated to both ends of a target nucleic acid molecule, and then this complex is used according to the methods described below. In certain embodiments, it is not necessary to include n-mers on both adapter ends, however, it is more convenient because it means that one does not have to use two different types of adaptors and then select for ligated fragments that have one of each type rather than two of one type. The ability to determine which strand is which is still possible in the situation wherein only one of the two adaptors has a double-stranded molecular barcode sequence.

The signer ligation adaptor may be any suitable ligation adaptor that is complementary to a ligation adaptor added to a double-stranded target nucleic acid sequence including, but not limited to a T-overhang, an A-overhang, a CG overhang, a blunt end, or any other ligatable sequence. In some embodiments, the signer ligation adaptor may be made using a method for A-tailing or T-tailing with polymerase extension; creating an overhang with a different enzyme; using a restriction enzyme to create a single or multiple nucleotide overhang, or any other method known in the art.

According to the embodiments described herein, the signer adaptor molecule may include at least two PCR primer or “flow cell” binding sites: a forward PCR primer binding site (or a “flow cell 1” (FC1) binding site); and a reverse PCR primer binding site (or a “flow cell 2” (FC2) binding site). The signer adaptor molecule may also include at least two sequencing primer binding sites, each corresponding to a sequencing read. Alternatively, the sequencing primer binding sites may be added in a separate step by inclusion of the necessary sequences as tails to the PCR primers, or by ligation of the needed sequences. Therefore, if a double-stranded target nucleic acid molecule has a signer adaptor molecule ligated to each end, each sequenced strand will have two reads—a forward and a reverse read.

The sequences of the two duplex strands seen in the two sequence reads may then be compared, and sequence information and mutations will be scored only if the sequence at a given position matches in both of the reads.

In some aspects of some embodiments, deliberate ligation of “U-shaped” adaptors or hairpin linkers containing 1) a double-stranded n-mer plus 2) primer binding sites to both ends of a captured fragment may be desirable. Producing closed circles of captured material may help facilitate removal of non-captured DNA by exonuclease digestion given that circularized DNA will be protected from digestion by such enzymes. Additionally, closed circles may be pre-amplified using rolling circle amplification or serve as the substrate for continuous loop sequencing. Recognition sites for restriction endonuclease digestion could be engineered into these adaptors to render closed loops open once again if more convenient for subsequent steps.

In another embodiment, flow cell sequences or PCR binding sites, again denoted as FC1 and FC2, may be included in both the PCR primers and the hairpin linker adaptor, as well as a ligatable sequence on the end of the hairpin linker (denoted as L below). The hairpin linker adaptor may additionally include one or more cleavable sequences, denoted as R in the example below (the R may be any appropriate restriction enzyme target sequence, or any other cleavable sequence). Such a hairpin linker design is shown below:

These products may then be sequenced directly. This design has the advantage of allowing for targeted sequencing of a specific region of the genome, and furthermore avoids the need to sequence a hairpin product, as sequencing of a hairpin will be less efficient due to the self-complementarity present within the hairpin molecule.

Following PCR duplication of the product and formation of consensus reads based upon the DMI sequence among all the PCR duplicates, the sequences of the two strands (denoted DNA and DNA′) can then be compared to form a duplex consensus sequence.

The signer adaptor molecules contain ligatable ends to allow attachment of the adaptor to a target DNA molecule. In some embodiments, the ligatable end may be complementary to a DNA overhang on the target DNA, for example, one generated by digestion of target DNA with a restriction endonuclease. Selective ligation of the adaptor to the targeted DNA containing the matching Single-stranded overhanging DNA sequence will then allow for partial purification of the targeted DNA. In some embodiments, the signer adaptor molecule, or a hairpin linker signer adaptor molecule, may additionally contain modifications such as biotin to facilitate affinity purification of target DNA that has ligated to the adaptor.

In another embodiment, specific PCR primers can selectively amplify specific regions of genome when the adaptor that is ligated to the other end of the molecule is a hairpin (or “U-shape”). Alternatively, this method may be used with or without the need for this cleavable hairpin sequence. Preparation of DNA for double strand error correction may be performed by PCR amplification in a hairpin structure

Another embodiment involves fragmentation of DNA at defined regions, for example by treatment of DNA with a site-specific restriction endonuclease or a mixture of such endonudeases, followed by annealing of a hairpin oligonucleotide linker, and amplification of the hairpin complex with PCR primers sufficient for amplification of the desired DNA sequence. Annealing of the hairpin linker to only one of the two ends of the DNA duplex could be accomplished by using different restriction enzymes to cut on either end of the target duplex, and then having the hairpin linker ligation adaptor being ligatable to only one of the two resultant ligatable ends.

This product can then be subjected to sequencing and error correction using DMI. The DMI sequence allows one to group together products of PCR amplification arising from a single molecule of duplex DNA. The sequences of the two DNA strands can then be compared for error correction.

The DMI method described herein have several uses. In some embodiments, the DMI method described herein may be used in methods to obtain the sequence or other sequence-related information of a double-stranded target nucleic acid molecule. According to the embodiments described herein, the term “double-stranded target nucleic acid molecule” includes a double-stranded DNA molecule or a double-stranded RNA molecule. Thus, the DMI methods of use described herein are applicable to genotyping and other applications related to sequencing of DNA molecules, but are also applicable to RNA sequencing applications such as for sequencing of double-stranded RNA viruses. Methods for sequencing RNA may include any of the embodiments described herein with respect to DNA sequencing, and vice-versa. For example, any double stranded target nucleic acid molecule may be ligated to a pool of signer adaptor molecule which includes a double-stranded RNA or DNA n-mer tag and an RNA or DNA ligation adapter as described above. Methods exist for directly sequencing RNA; alternatively, the ligated product may be reverse transcribed into DNA, and then sequenced as a double-stranded target DNA molecule.

In one embodiment, the double-stranded target nucleic acid molecule may be a sheared double-stranded DNA or RNA fragment. The sheared target DNA or RNA molecule may be end repaired and a double-stranded target nucleic acid sequence ligation adaptor may be added to each end of the sheared target DNA or RNA molecule. The double-stranded target nucleic acid sequence ligation adaptor may be any suitable ligation adaptor that is complementary to the signer ligation adaptor described above including, but not limited to a T-overhang, an A-overhang, a CG overhang, blunt end or any other ligatable sequence. In some embodiments, the double-stranded target nucleic acid sequence ligation adaptor may be made using a method for A-tailing or T-tailing with polymerase extension; adding an overhang with a different enzyme; using a restriction enzyme to create a ligatable overhang; or any other method known in the art.

Methods to obtain the sequence or other sequence-related information of a double-stranded target nucleic acid molecule may include a step of ligating the double-stranded target nucleic acid molecule to at least one signer adaptor molecule, such as those described above, to form a double-stranded target nucleic acid complex. In one embodiment, each end of the double-stranded target nucleic acid molecule is ligated to a signer adaptor molecule. The double-stranded target nucleic acid complex is then amplified by a method known in the art (e.g., a PCR or non-PCR method known in the art), resulting in a set of uniquely labeled, amplified signer-target nucleic acid products. These products are then sequenced using any suitable method known in the art including, but not limited to, the Illumina sequencing platform, ABI SOliD sequencing platform, Pacific Biosciences sequencing platform, 454 Life Sciences sequencing platform, Ion Torrent sequencing platform, Helicos sequencing platform, and nanopore sequencing technology.

In certain embodiments, a method of generating an error corrected double-stranded consensus sequence is provided. Such a method, also referred to as DMI-based double strand error correction (DDSEC), allows for a quantitative detection of sites of DNA damage. DDSEC facilitates the detection of DNA damage signatures, in that single stranded DNA mutations that are not present in the complementary strand can be inferred to be artifactual mutations arising from damaged nucleotides. Not only can one correct for these erroneous mutations, but the ability to indirectly infer that damage is present on the DNA could be a useful biomarker (e.g. for cancer risk, cancer metabolic state, mutator phenotype related to defective damage repair, carcinogen exposure, chronic inflammation exposure, individual-specific aging, neurodegenerative diseases etc.). The ability to use different polymerases during the first round(s) of PCR to mis-incorporate at damage sites could potentially add even more information. Besides polymerases, other DNA modifying/repair enzymes could be used prior to amplification to convert damage of one sort that doesn't give a specific mutagenic signature into another sort that does with whatever polymerase is used. Alternatively, DNA modifying/repair enzymes could be used to remove damaged bases, and one could sequence both strands of DNA both with and without the enzymatic treatment. Mutations in single-stranded DNA that are seen to be removed by the enzymatic treatment can thus be inferred to be arising due to DNA damage. This could be useful on human nuclear or mtDNA but also might also be useful with model organisms (mice, yeast, bacteria etc), treated with different new damaging agents, facilitating a screen for DNA damaging compounds that would be analogous to the widely used Ames test.

The method of generating an error corrected double-stranded consensus sequence may include a first stage termed “DMI-based single strand error correction” (DSSEC) followed by a second stage of double strand error correction (DDSEC). Therefore, the method includes steps of tagging individual duplex DNA molecules with a signer adaptor molecule, such as those described above; generating a set of PCR duplicates of the tagged DNA molecules by performing a suitable PCR method; creating a single strand consensus sequence from all of the PCR duplicates which arose from an individual molecule of single-stranded DNA. Each DNA duplex should result in two single strand consensus sequences. The error correction through these three steps conclude the first stage and is termed DSSEC.

The method of generating an error corrected double-stranded consensus sequence further comprises the second stance that is termed DMI-based double strand error correction. The double strand error correction includes steps of comparing the sequence of the two single strand consensus sequences arising from a single duplex DNA molecule, and further reducing sequencing or PCR errors by considering only sites at which the sequences of both single-stranded DNA molecules are in agreement. The method that includes the first stage and the second stage termed double strand error correction.

The step of tagging of both strands of individual duplex DNA is accomplished by randomly ligation a pool of signer adapter with fixed sequence; as the complementary nature of the two strands of such a tag sequence allows the two molecules to be grouped together for error correction. Alternatively, as described above, the two duplex DNA strands may be linked by ligation of a U-shaped molecular barcode adaptor molecule, and the two DNA strands can thus both be tagged with a single-stranded molecular barcode tag.

In the method described above, a set of sequenced signer-DNA products generated in the methods described above may be grouped into families of paired target nucleic acid strands based on a common set of DMI. Then, the paired target nucleic acid strands can be filtered to remove nucleotide positions where the sequences seen on both of the paired partner DNA strands are not complementary. This error corrected double-stranded consensus sequence may be used in a method for confirming the presence of a true mutation (as opposed to a PCR error or other artifactual mutation) in a target nucleic acid sequence. According to certain embodiments, such a method may include identifying one or more mutations present in the paired target nucleic acid strands that have one or more nucleotide positions that disagree between the two strands, then comparing the mutation present in the paired target nucleic acid strands to the error corrected double-stranded consensus sequence. The presence of a true mutation is confirmed when the mutation is present on both of the target nucleic acid strands and also appear in all members of a paired target nucleic acid family.

The accuracy of current approaches to next-generation sequencing is limited due to their dependence on interrogating single-stranded DNA. This dependence makes potential sources of error such as PCR amplification errors and DNA damage fundamentally limiting. However, the complementary strands of a double-stranded DNA molecule (or “DNA duplex”) contain redundant sequencing information (i.e., one molecule reciprocally encoding the sequence information of its partner) which can be utilized to eliminate such artifacts. Limitations related to sequencing single-stranded DNA (e.g., sequencing errors) may therefore be overcome using the methods described herein. This is accomplished by individually tagging and sequencing each of the two strands of a double-stranded (or duplex) target nucleic acid molecule and comparing the individual tagged amplicons derived from one half of a double-stranded complex with those of the other half of the same molecule. Double strand error correction (DDSEC), significantly lowers the error rate of sequencing. In some embodiments, the DDSEC method may be used in methods for high sensitivity detection of rare mutant and variant DNA as described further below.

DNA damage should not be a limiting factor in DDSEC, because miscoding damage events at a single base-pair position occur essentially exclusively on only one of the two DNA strands. For DNA damage to result in an artifactual mutation in DDSEC, damage would need to be present at the same nucleotide position on both strands. Even if complementary nucleotides in a duplex were both damaged, the damage would need to result in complementary sequencing errors to result in mis-scoring of a mutation. Likewise, spontaneous PCR errors would need to result in complementary mutations at the same position on both strands.

According to some embodiments, the sequencing method may be performed using the Illumina or similar platforms including those enumerated above without the use of signer adaptor molecules, therefore a DMI can only be based on the sequence information of the targets, such as random shear points of DNA, as identifiers. For a given DNA sequence seen in sequencing read 1 with a specific set of shear points, the partner strand will be seen as a matching sequence in read two with identical shear points. In practice, this approach is limited by the limited number of possible shear points that overlap any given DNA position. However, according to some embodiments, shear points of a target nucleic acid molecule may be used as unique identifiers to identify double-stranded (or duplex) pairs, resulting in an apparent error frequency at least as low as that seen with traditional sequencing methods, but with a significantly lower loss of sequence capacity. In other embodiments, DDSEC based on shear points alone may have a role for confirmation that specific mutations of interest are true mutations which were indeed present in the starting sample (i.e. present in both DNA strands), as opposed to being PCR or sequencing artifacts. Use of DMI significantly reduced the complexity of deplex-seq experiment by using regular molecular adaptors with fix barcode sequence.

In certain embodiments, the DMI methods may also be used in methods of single-molecule counting for accurate determination of DNA or RNA copy number. Again, since the DMI calculated using the information of the molecular barcode in the signer adaptors, there are no altered steps required in library preparation, which is in contrast to other methods for using random tags for single-molecule counting. Single-molecule counting has a large number of applications including, but not limited to, accurate detection of altered genomic copy number (e.g., for sensitive diagnosis of genetic conditions such as trisomy 21), for accurate identification of altered mRNA copy number in transcriptional sequencing and chromatin immunoprecipitation experiments, quantification of circulating microRNAs, quantification of viral load of DNA or RNA viruses, quantification of microorganism abundance, quantification of circulating neoplastic cells, counting of DNA-labeled molecules of any variety including tagged antibodies or aptamers, and quantification of relative abundances of different individual's genomes in forensic applications.

In another embodiment, the DMI may be used in methods for unambiguous identification of PCR duplicates. In order to restrict sequencing analysis to uniquely sequenced DNA fragments, many sequencing studies include a step to filter out PCR duplicates by using the shear points at the ends of DNA molecules to identify distinct molecules. When multiple molecules exhibit identical shear points, all but one of the molecules are discarded from analysis under the assumption that the molecules represent multiple PCR copies of the same starting molecule. However, sequence reads with identical shear points can also reflect distinct molecules because there are a limited number of possible shear points at any given genomic location, and with increasing sequencing depth, recurrent shear points are increasingly likely to be seen. Through taking the molecular barcode information of the signer adaptor into consideration, DMI allows every molecule to be uniquely identified, true PCR duplicates may be unambiguously identified by virtue of having a common (i.e., the same or identical) DMI. This approach would thereby minimize the loss of data by overcoming the intrinsic limitations of using shear points to identify PCR duplicates.

Importantly, DMI methods allow using standard sequencing adaptors. Thus, use of DDSEC does not require any significant deviations from the normal workflow of sample preparation for Illumina DNA sequencing. Moreover, the DDSEC approach can be generalized to nearly any sequencing platform because DMI can be computed from any signer nucleotide and target nucleotide. The compatibility of DMI with existing sequencing workflows, the potential for greatly reducing the error rate of DNA sequencing, and the multitude of applications for the DMI validate DDSEC as a technique that may play a general role in next generation DNA sequencing.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Example 1 Generation of Signer Nucleotides, DMI and Their Use in Sequencing Double Stranded Target DNA

### Material and Methods

Oligonucleotides were from IDT and were ordered as PAGE purified. Klenow exo-was from NEB. T4 ligase was from Enzymatics. DNA: Multiplex I cfDNA Reference Standard were purchased from Horizon.

Signer nucleotides (adaptors). The signer nucleotides were synthesized from two oligos, designated as:

the plus strand:

(SEQ ID NO: 1)
AATGATACGG CGACCACCGA GATCTACACT CTTTCCCTAC
ACGACGCTCT TCCNNNNNNN NNNNNGATCT;

and

the minus strand:

(SEQ ID NO: 2)
ACTGNNNNNN NNNNNNAGAT CGGAAGAGCA CACGTCTGAA
CTCCAGTCAC.

The denotes a molecular barcode with 12 nucleic acid in length. The molecular barcode in minus strand are reverse complementary with the molecular barcode in plus strand.

The sequence is one of follows:

(SEQ ID NO: 3)
TCCCTTGTCTCC,
(SEQ ID NO: 4)
ACGAGACTGATT,
(SEQ ID NO: 5)
GCTGTACGGATT,
(SEQ ID NO: 6)
ATCACCAGGTGT,
(SEQ ID NO: 7)
TGGTCAACGATA,
(SEQ ID NO: 8)
ATCGCACAGTAA,
(SEQ ID NO: 9)
GTCGTGTAGCCT,
(SEQ ID NO: 10)
AGCGGAGGTTAG,
(SEQ ID NO: 11)
ATCCTTTGGTTC,
(SEQ ID NO: 12)
TACAGCGCATAC,
(SEQ ID NO: 13)
ACCGGTATGTAC,
(SEQ ID NO: 14)
AATTGTGTCGGA,
(SEQ ID NO: 15)
TGCATACACTGG,
(SEQ ID NO: 16)
AGTCGAACGAGG,
(SEQ ID NO: 17)
ACCAGTGACTCA,
(SEQ ID NO: 18)
GAATACCAAGTC,
(SEQ ID NO: 19)
GTAGATCGTGTA,
(SEQ ID NO: 20)
TAACGTGTGTGC,
(SEQ ID NO: 21)
CATTATGGCGTG,
(SEQ ID NO: 22)
CCAATACGCCTG,
(SEQ ID NO: 23)
GATCTGCGATCC,
(SEQ ID NO: 24)
CAGCTCATCAGC,
(SEQ ID NO: 25)
CAAACAACAGCT,
(SEQ ID NO: 26)
GCAACACCATCC,
(SEQ ID NO: 27)
GCGATATATCGC,
(SEQ ID NO: 28)
CGAGCAATCCTA,
(SEQ ID NO: 29)
AGTCGTGCACAT,
(SEQ ID NO: 30)
GTATCTGCGCGT,
(SEQ ID NO: 31)
CGAGGGAAAGTC,
(SEQ ID NO: 32)
CAAATTCGGGAT,
(SEQ ID NO: 33)
AGATTGACCAAC,
(SEQ ID NO: 34)
AGTTACGAGCTA,
(SEQ ID NO: 35)
GCATATGCACTG,
(SEQ ID NO: 36)
CAACTCCCGTGA,
(SEQ ID NO: 37)
TTGCGTTAGCAG,
(SEQ ID NO: 38)
TACGAGCCCTAA,
(SEQ ID NO: 39)
CACTACGCTAGA,
(SEQ ID NO: 40)
TGCAGTCCTCGA,
(SEQ ID NO: 41)
ACCATAGCTCCG,
(SEQ ID NO: 42)
TCGACATCTCTT,
(SEQ ID NO: 43)
GAACACTTTGGA,
(SEQ ID NO: 44)
GAGCCATCTGTA,
(SEQ ID NO: 45)
TTGGGTACACGT,
(SEQ ID NO: 46)
AAGGCGCTCCTT,
(SEQ ID NO: 47)
TAATACGGATCG,
(SEQ ID NO: 48)
TCGGAATTAGAC,
(SEQ ID NO: 49)
TGTGAATTCGGA,
(SEQ ID NO: 50)
CATTCGTGGCGT,
(SEQ ID NO: 51)
TACTACGTGGCC,
(SEQ ID NO: 52)
GGCCAGTTCCTA,
(SEQ ID NO: 53)
GATGTTCGCTAG,
(SEQ ID NO: 54)
CTATCTCCTGTC,
(SEQ ID NO: 55)
ACTCACAGGAAT,
(SEQ ID NO: 56)
ATGATGAGCCTC,
(SEQ ID NO: 57)
GTCGACAGAGGA,
(SEQ ID NO: 58)
TGTCGCAAATAG,
(SEQ ID NO: 59)
CATCCCTCTACT,
(SEQ ID NO: 60)
TATACCGCTGCG,
(SEQ ID NO: 61)
AGTTGAGGCATT,
(SEQ ID NO: 62)
ACAATAGACACC,
(SEQ ID NO: 63)
CGGTCAATTGAC,
(SEQ ID NO: 64)
GTGGAGTCTCAT,
(SEQ ID NO: 65)
GCTCGAAGATTC,
(SEQ ID NO: 66)
AGGCTTACGTGT,
(SEQ ID NO: 67)
TCTCTACCACTC,
(SEQ ID NO: 68)
ACTTCCAACTTC,
(SEQ ID NO: 69)
CTCACCTAGGAA,
(SEQ ID NO: 70)
GTGTTGTCGTGC,
(SEQ ID NO: 71)
CCACAGATCGAT,
(SEQ ID NO: 72)
TATCGACACAAG,
(SEQ ID NO: 73)
GATTCCGGCTCA,
(SEQ ID NO: 74)
CGTAATTGCCGC,
(SEQ ID NO: 75)
GGTGACTAGTTC,
(SEQ ID NO: 76)
ATGGGTTCCGTC,
(SEQ ID NO: 77)
TAGGCATGCTTG,
(SEQ ID NO: 78)
AACTAGTTCAGG,
(SEQ ID NO: 79)
ATTCTGCCGAAG,
(SEQ ID NO: 80)
AGCATGTCCCGT,
(SEQ ID NO: 81)
GTACGATATGAC,
(SEQ ID NO: 82)
GTGGTGGTTTCC,
(SEQ ID NO: 83)
TAGTATGCGCAA,
(SEQ ID NO: 84)
TGCGCTGAATGT,
(SEQ ID NO: 85)
ATGGCTGTCAGT,
(SEQ ID NO: 86)
GTTCTCTTCTCG,
(SEQ ID NO: 87)
CGTAAGATGCCT,
(SEQ ID NO: 88)
GCGTTCTAGCTG,
(SEQ ID NO: 89)
GTTGTTCTGGGA,
(SEQ ID NO: 90)
GGACTTCCAGCT,
(SEQ ID NO: 91)
CTCACAACCGTG,
(SEQ ID NO: 92)
CTGCTATTCCTC,
(SEQ ID NO: 93)
ATGTCACCGCTG,
(SEQ ID NO: 94)
TGTAACGCCGAT,
(SEQ ID NO: 95)
AGCAGAACATCT,
(SEQ ID NO: 96)
TGGAGTAGGTGG,
(SEQ ID NO: 97)
TTGGCTCTATTC,
(SEQ ID NO: 98)
GATCCCACGTAC.

Totally 96 pairs of signer nucleotide were synthesized. Each pair of adaptor strands were annealed by combining equimolar amounts of each oligo to a final concentration of 50 micromolar and heating to 95° C. for 5 minutes.

Total 96 annealed adaptors are pooled equimolarly, thereby generate a pool of signer nucleotides.

Sequencing library preparation. 5 ng of cfDNA was end-repair with the NEB end-repair kit per the manufacturer's protocol, then T-tailed in a reaction containing 5 units Klenow exo-, 1 mM dTTP, 50 mM NaCl, 10 mM Tris-HCI pH 7.9, 10 mM MgCl₂, 1 mM. The reaction proceeded for 1 hour at 37 C. DNA was purified with 1 volumes of AMPure XP beads.

The T-tailed DNA was then ligated with 250 pmol adaptors in a reaction containing 3000 units T4 DNA ligase, 50 mM Tris-HCI pH 7.6, 10 mM MgCl₂, 5 mM DTT, 1 mM ATP. The reaction was incubated 25° C. for 15 minutes, and purified with 1 volumes of AMPure XP beads.

Pre-capture amplification. Signer-target DNA was PCR amplified with primers AATGATACGG CGACCACCGA G (SEQ ID NO: 99) and GTGACTGGAG TTCAGACGTG TGC (SEQ ID NO: 100) using the Kappa high-fidelity PCR kit for 12 cycles with an annealing temperature of 60° C. The product was purified with 1 volume of AMPure XP beads.

DNA capture. Target capture was performed with the Agilent SureSelect system per the manufacturer's recommendations. The capture set targeted an arbitrary 80 kb region of the genome consisting of coding sequences of cancer related genes. Capture baits were 120 nt in length, and were prepared with the Agilent eArray tool with 3× tiling.

Post-capture amplification. Captured DNA was amplified with PCR primers AATGATACGGCGACCACCGAG (SEQ ID NO: 99) and CAAGCAGAAG ACGGCATACGAGATNNNNGTGACT GGAGTTCAGA CGTGTGC (SEQ ID NO: 101) where indicates the position of a fixed multiplexing barcode sequence). 1.5 pm of DNA was used for sequencing on a NextSeq500.

Data processing. Reads with intact signer adaptors include known 12 bp molecule barcodes. These reads were identified by filtering out reads that lack the expected fixed sequence of molecule barcode. The DMI for each read was computed by taking the molecule barcode sequence of the forward and reverse sequencing reads, and sequence at position 5-17 of the target nucleotide sequence. The DMI was then computationally added to the read header, and the signer adapter sequence removed. The first 4 nucleotides located following the adaptor sequence were also removed due to the propensity for ligation and end-repair errors to result in an elevated error rate near the end of the DNA fragments. Reads having common (i.e., identical) DMI sequences were grouped together, and were collapsed to generate a consensus read. Sequencing positions were discounted if the consensus group covering that position consisted of fewer than 3 members, or if fewer than 90% of the sequences at that position in the consensus group had the identical sequence. Reads were aligned to the human genome with the Burrows-Wheeler Aligner (BWA). The consensus sequences were then paired with their strand-mate by grouping each 48 nucleotide tag of form AabB in read 1 with its corresponding tag of form BbaA in read 2. Resultant sequence positions were considered only when information from both DNA strands was in perfect agreement. An overview of the data processing workflow is as follows:

1. Discard reads that do not have the 12 nt molecular barcode sequence.

2. Compute DMI tags from molecule barcode and target nucleotide of read 1 and read 2, and transfer the combined 48 nt DMI sequence into the read header.

3. Remove the 5 nt fixed reference sequence.

4. Trim an additional 4 nt from the 5′ ends of each read pair.

5. Group together reads which have identical 48 nt DMI.

6. Collapse to DMI consensus reads, scoring only positions with 3 or more DMI duplicates and >90% sequence identity among the duplicates.

7. For each read in read 1 file having DMI of format AabB, group with corresponding DCS partner in read 2 with DMI of format BbaA.

8. Only score positions with identical sequence among both DCS partners.

9. Align reads to the human genome.

Code for carrying out the workflow may be pre-existing or may involve programming within the skill of those in the art.

### Overview

To overcome limitations in the sensitivity of variant detection by single-stranded next-generation DNA sequencing, an alternative approach to library preparation and analysis was designed, which is known herein as DMI-based Double Strand Error Correction (DDSEC) method (FIG. 1). The DDSEC method described herein involves tagging both strands of duplex DNA with a signer nucleotide, which is a complementary double-stranded molecular barcode with known sequence. When randomly ligating a pool of the signer nucleotides with target nucleotides, a pool of unique signer-target complex was created, each supposed to have a unique DMI. The signer-target molecules are then PCR amplified. Every duplicate that arises from a single strand of DNA will have the same DMI, and thus each strand in a DNA duplex pair generates a distinct, yet related population of PCR duplicates after amplification owing to the complementary nature of the DMI on the two strands of the duplex. Comparing the sequence obtained from each of the two strands comprising a single molecule of duplex DNA facilitates differentiation of sequencing errors from true mutations. When an apparent mutation is, due to a PCR or sequencing error, the substitution will only be seen on a single strand. In contrast, with a true DNA mutation, complementary substitutions will be present on both strands.

Following tagging with double-stranded signer nucleotides, PCR amplification and sequencing, a family of molecules is obtained that arose from a single DNA molecule; members of the same PCR “family” are then grouped together by virtue of having a common (i.e., the same) DMI. The sequences of uniquely tagged PCR duplicates are subsequently compared in order to create a PCR consensus sequence. Only DNA positions that yield the same DNA sequence in a specified proportion of the PCR duplicates in a family, such as 90% of the duplicates in one embodiment, are used to create the PCR consensus sequence. This step filters out random errors introduced during sequencing or PCR to yield the PCR consensus sequences, each of which derives from an individual molecule of single-stranded DNA. This method is called DMI-based single strand error correction (DSSEC).

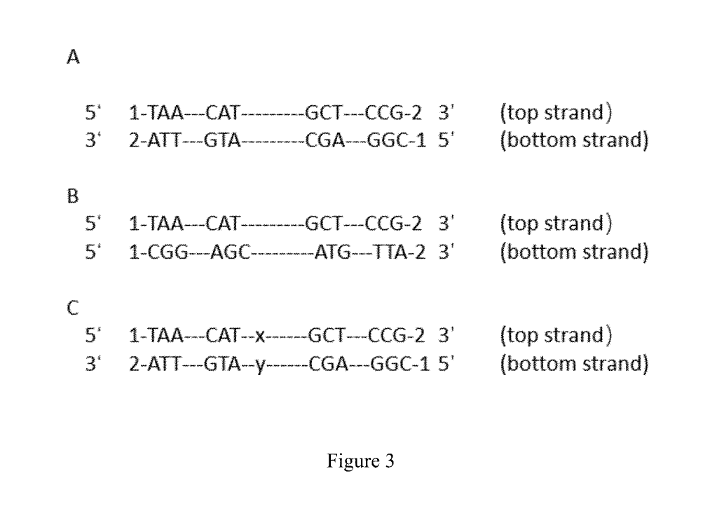

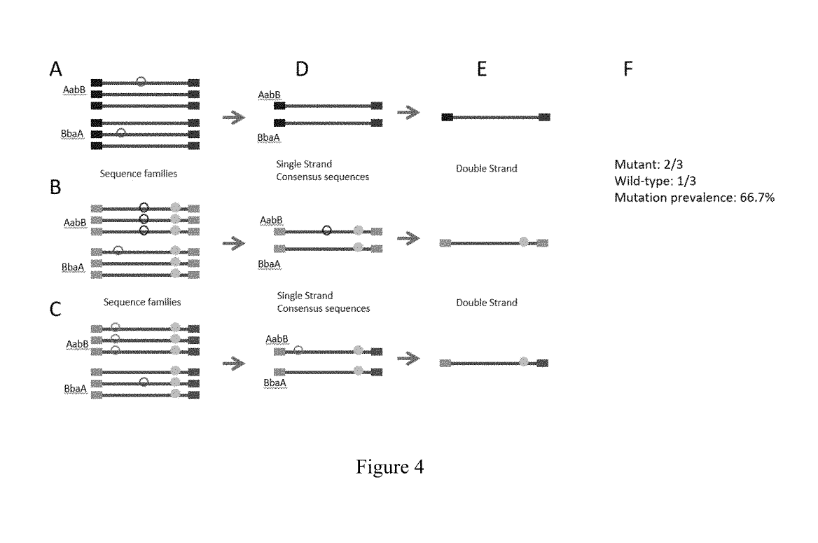

Next, PCR consensus sequences arising from two complementary strands of duplex DNA can be identified by virtue of the complementary DMI (FIG. 3) to identify the “partner DMI.” Specifically, a 48-nucleotide DMI consists of four 12-nucleotide sequences that can be designated AabB. For an DMI of form AabB in read 1, the partner DMI will is BbaA in read 2. An example to illustrate this point is given in FIG. 4. Following partnering of two strands by virtue of their complementary DMI, the sequences of the strands are compared. Sequence reads at a given position are kept only if the read data from each of the two paired strands is in agreement.

### Results

In order to generate a unique signature for each of the strands of duplex DNA, signer nucleotide (adaptor) with the standard sequences required for the Illumina system were synthesized. The signer adaptor having a molecular barcode n-mer that is 12 nucleotides.

Multiplex I cfDNA Reference Standard were purchased from Horizon. DNA for sequencing end-repaired by standard methods. Standard Illumina library preparation protocols involve ligating A-tailed DNA to T-tailed adaptors. However, because A-tailed adaptors were used, the DNA was T-tailed by incubating the end-repaired DNA with Klenow exo-DNA polymerase and 1 mM dTTP. The adaptor-ligated library was PCR amplified and subjected to SureSelect capture, with targeting of an arbitrary 800 kb portion of the genome (DNA coordinates available upon request). The efficiency of adaptor ligation, PCR amplification, DNA capture, and sequencing were comparable to those seen with standard library preparation methods (data not shown). Although Agilent Sure Select probes are used in this example, any suitable method of DNA selection may be used to capture particular target double-stranded DNA sequences. For example, selection and capture may be accomplished by any selection by hybridization method (e.g., Agilent SureSelect, Primer Extension Capture, exploitation of biotinylated PCR amplicons as bait, Agilent HaloPlex) wherein probes that target the desired double-stranded DNA sequence may be recovered by an in-array capture (using probes immobilized on glass slides) or by affinity using magnetic beads in an in-solution capture. In addition, mitochondrial and some other forms of DNA may be isolated by size selection. Alternatively, in some embodiments, no enrichment is performed.

Mutations were initially scored without consideration of the DMI. PCR duplicates were filtered out with samtools rmdup, a standard tool which uses the shear points of DNA molecules to identify PCR duplicates, as molecules arising from duplicated DNA will have shared shear points. In order to focus specifically on non-clonal mutations, only those positions in the genome with at least 20× coverage and at which fewer than 5% of reads differed from the hg19 reference sequence were considered. This approach resulted in 80.1 million nucleotides of sequence data and 56,780 mutations, indicating an overall mutation frequency of 7.01×10-4, in accord with the error rate of Illumina next-generation sequencing of −0.1-1%.

Next, the DMI information was used to group together PCR duplicates that arose from individual single-stranded DNA molecules and to create a consensus sequence from the family of duplicates. At least 3 PCR duplicates were required, with at least 90% agreement in sequence among all duplicates, to consider a site for mutations. Scoring the mutation frequency as above, again considering only sites with a minimum of 20× coverage and with <5% of reads differing from reference, resulted in 150 million nucleotides of sequence with 7,050 mutations and an overall mutation frequency of 4.7×10-5, consistent with prior reports. Notably, far more nucleotides of DNA sequence were obtained in this approach (150 million) than in the standard Illumina sequencing approach (80.1 million) detailed above which is dependent on use of the shear points of single-ended reads to identify PCR duplicates. The improved sequence coverage arose from use of the DMI to identify PCR duplicates, because identifying PCR duplicates by consideration of uniquely sheared DNA ends is fundamentally limited by the small number of possible shear points that overlap a given position of the genome and the propensity for specific genomic regions to be more readily undergo shearing. Thus filtering PCR duplicates by using shear points resulted in discarding a large portion of the reads.

Finally, the complementary nature of the double-stranded DMI sequences was used to identify pairs of consensus groups that arose from complementary DNA strands. Sequence reads were considered only when the read data from each of the two strands is in perfect agreement. In a pilot experiment, after grouping of PCR duplicates as above, 30,560 DMI partner pairs were found, indicative that fewer than 1% of tags had their corresponding partner tag present in the library. The low recovery of tag pairs was most likely due to inadequate amplification of the starting DNA library. Among these tag-pairs, 23,658 duplex consensus strands were identified with an average strand length of 82 nucleotides, resulting in 2.3 million nucleotides of DNA consensus sequence. The sequences of the paired duplex strands disagreed at 3348 of the nucleotide positions, indicative of single-stranded errors (i.e. PCR or sequencing errors); these sites of disagreement were removed, leaving only bases at which the sequence of both duplex strands was in perfect agreement. Next, as above, analysis of mutation frequencies was restricted to sites with at least 10× coverage and at which fewer than 10% of reads disagreed from the hg19 reference sequence. Because the 2.3 million nucleotides of read data were spread across a 800 kb target, our average depth was only ˜3×. Thus only 15,436 nucleotides of DNA sequence corresponded to sites with at least 10× depth. Among these sites, zero mutations were seen. To increase the number of tag pairs considered, analysis described above was repeated, but PCR duplicates were grouped with a minimum of only 1 duplicate per site. This resulted in 30,439 nucleotides of DNA sequence with at least 10× depth. Again, no mutations were detected.

Current experiments are being performed on vastly smaller target DNA molecules (ranging from −300 bp to −20 kb in size). Use of smaller DNA targets will allow for much greater sequencing depth, and far more accurate assessment of the background mutation rate of the assay. In addition, the protocol has been modified to incorporate a greater number of PCR cycles initiated off a smaller number of genome equivalents, which will increase the fraction of tags for which both of the partner tag strands have been sufficiently amplified to be represented in the final sequence data. Indeed, among the 3.2 million DMIs present in our initial library which underwent PCR duplication, 1.2 million of the DMIs were present only once, indicating insufficient amplification of the DNA due in part to the low number of PCR cycles used.

1. A method to generate DMI (Digital Molecular Identifier) for sequencing read comprising:
obtaining a pool of signer nucleotides;
mixing the signer nucleotides with target nucleotides;
performing a reaction of adding the signer nucleotides to the target nucleotides to form signer-target nucleotides complexes;
amplifying the signer-target nucleic acid complexes, resulting in a set of amplified signer-target nucleotides complexes;
sequencing the amplified signer-target nucleotides complexes; and
combining the information of the signer nucleotides, the target nucleotides, and the signer-target nucleotides complexes.

2. The method of claim 1, wherein the signer nucleotides are adaptors with different molecular barcodes.

3. The method of claim 1, wherein the reaction is a ligation reaction allowing the signer nucleotides to be randomly ligated to the target nucleotides.

4. The method of claim 2, wherein the molecular barcodes are a single strand or double strand.

5. The method of claim 1, wherein the information includes sequence information, length of the target nucleotides and the location of the target nucleotides on a reference genome.

6. A sequencing library preparation kit allowing computing DMI (Digital Molecular Identifier), comprising:
a pool of nucleotides with known sequence serve as signer nucleotides; and
reagents that allow the signer nucleotides to be randomly added to target nucleotides, thereby generating a molecular signature.

7. The sequencing library preparation kit of claim 6, wherein the signer nucleotides are adaptors with a molecular barcode of at least 3 nucleotides in length.

8. The sequencing library preparation kit of claim 6, wherein the target nucleotides are double-stranded DNA or RNA molecules.

9. The sequencing library preparation kit of claim 6, wherein the signer nucleotides include at least two PCR primer binding sites, at least two sequencing primer binding sites, or both.

10. The sequencing library preparation kit of claim 6, wherein the signer nucleotides are added to the target nucleotides via a ligation reaction.

11. The sequencing library preparation kit in claim 6, wherein the signer nucleotides include a ligation adaptor selected from the group consisting of a T-overhang, an A-overhang, a CG overhang, a blunt end, and a ligatable nucleic acid sequence.

12. The sequencing library preparation kit in claim 6, wherein the signer nucleotides are Y-shaped, U-shaped, or a combination thereof.

13. The sequencing library preparation kit in claim 6, further comprising a module to compute DMI by using the information of the signer nucleotides and target nucleotides, wherein the information includes sequence information, the length of the target nucleotides and the location of the target nucleotides on a reference genome.

14. A method of obtaining the sequence of a double-stranded target nucleic acid comprising:
obtaining a pool of double-stranded signer nucleotides;
mixing the double-stranded signer nucleotides with double-stranded target nucleotides;
performing a reaction allowing the double-stranded signer nucleotides to be randomly added to double-stranded target nucleotides to form double-stranded signer-target nucleotides complexes;
amplifying the double-stranded signer-target nucleotides complexes, resulting in a set of amplified signer-target nucleotides complexes; and
sequencing the amplified double-stranded signer-target nucleotides complexes.

15. The method in claim 14, wherein the double-stranded target nucleotides are double-stranded DNA or RNA molecules.

16. The method in claim 14, further comprising:
generating an error-corrected single-stranded consensus sequence by (i) generating a DMI (Digital Molecular Identifier) using the information of the double-stranded signer-target nucleotides complexes; (ii) grouping the sequenced amplified signer-target nucleotides products into families of target nucleic acid strands based on the DMI; and (ii) removing target nucleic acid strands having one or more nucleotide positions where paired target nucleic acid strands disagree, or removing nucleotide positions from nucleic acid strands where single strands disagree at a specific position.

17. The method of claim 14, wherein the double-stranded target nucleotides are double-stranded circulating tumor DNA or reverse transcribed circulating tumor RNA fragment.

18. The method of claim 14, wherein the double-stranded nucleotides include a double-stranded target nucleic acid sequence ligation adaptor.

19. The method of claim 18, wherein the double-stranded target nucleic acid sequence ligation adaptor is selected from the group consisting of a T-overhang, an A-overhang, a CG overhang, a blunt end, and a ligatable nucleic acid sequence.

20. The method of claim 14, wherein each end of the double-stranded target nucleotides is ligated to a signer adaptor molecule.

21. The method of claim 20, wherein the signer adaptor molecule includes a molecular barcode sequence and an adaptor; the molecular barcode sequence includes a degenerate or semi-degenerate nucleic acid sequence; and the adaptor allows the signer adaptor molecule to be ligated to the double-stranded target nucleotides.

22. The method of claim 14, wherein the double-stranded signer nucleotide includes at least two PCR primer binding sites, at least two sequencing primer binding sites, or a combination thereof.

23. A method of generating an error corrected sequence comprising:
obtaining a pool of signer nucleotides;
mixing the pool of signer nucleotides with target nucleotides;
performing a reaction allowing the signer nucleotides to be added to the target nucleotides to form signer-target nucleotides complexes;
generating a set of PCR duplicates of the signer-target nucleotides complexes by performing PCR;
sequencing the PCR duplicates;
generating a DMI using the information of the signer-target nucleotides complexes; and
creating a single strand consensus sequence using the DMI from the sequenced PCR duplicates which arose from an individual molecule of single-stranded DNA.

24. The method in claim 23, wherein the information includes one or more of the signer nucleotides and target nucleotides, the location of a target nucleotide on a reference genome, and the length of the target nucleotides.

25. The method in claim 23, further comprising:
comparing the sequence of two single strand consensus sequences arising from a single duplex DNA molecule; and
reducing sequencing or PCR errors by (i) grouping the sequenced signer-target nucleic acid products into families of paired target nucleic acid strands based on a common set of DMI; and (ii) removing paired target nucleic acid strands having one or more nucleotide positions where paired target nucleic acid strands disagree, or removing nucleotide positions from nucleic acid strands where the paired strands disagree at a specific position.

26. The method in claim 23, wherein the signer nucleotides include a molecular barcode having at least 3 nucleotides in length.


SEQUENCE LISTING (101 sequences)

SEQ ID NO: 1 - DNA, 70 bp, Artificial Sequence
  Synthetic oligonucleotide
  misc_feature (54)..(65): n is a, c, g, or t
aatgatacggcgaccaccgagatctacactctttccctacacgacgctcttccnnnnnnnnnnnngatct

SEQ ID NO: 2 - DNA, 50 bp, Artificial Sequence
  Synthetic oligonucleotide
  misc_feature (5)..(16): n is a, c, g, or t
gatcnnnnnnnnnnnnagatcggaagagcacacgtctgaactccagtcac

SEQ ID NO: 3 - DNA, 12 bp, Artificial Sequence
  Synthetic oligonucleotide
tcccttgtctcc

SEQ ID NO: 4 - DNA, 12 bp, Artificial Sequence
  Synthetic oligonucleotide
acgagactgatt

SEQ ID NO: 5 - DNA, 12 bp, Artificial Sequence
  Synthetic oligonucleotide
gctgtacggatt

SEQ ID NO: 6 - DNA, 12 bp, Artificial Sequence
  Synthetic oligonucleotide
atcaccaggtgt

SEQ ID NO: 7 - DNA, 12 bp, Artificial Sequence
  Synthetic oligonucleotide
tggtcaacgata

SEQ ID NO: 8 - DNA, 12 bp, Artificial Sequence
  Synthetic oligonucleotide
atcgcacagtaa

SEQ ID NO: 9 - DNA, 12 bp, Artificial Sequence
  Synthetic oligonucleotide
gtcgtgtagcct

SEQ ID NO: 10 - DNA, 12 bp, Artificial Sequence
  Synthetic oligonucleotide
agcggaggttag

SEQ ID NO: 11 - DNA, 12 bp, Artificial Sequence
  Synthetic oligonucleotide
atcctttggttc

SEQ ID NO: 12 - DNA, 12 bp, Artificial Sequence
  Synthetic oligonucleotide
tacagcgcatac

SEQ ID NO: 13 - DNA, 12 bp, Artificial Sequence
  Synthetic oligonucleotide
accggtatgtac

SEQ ID NO: 14 - DNA, 12 bp, Artificial Sequence
  Synthetic oligonucleotide
aattgtgtcgga

SEQ ID NO: 15 - DNA, 12 bp, Artificial Sequence
  Synthetic oligonucleotide
tgcatacactgg

SEQ ID NO: 16 - DNA, 12 bp, Artificial Sequence
  Synthetic oligonucleotide
agtcgaacgagg

SEQ ID NO: 17 - DNA, 12 bp, Artificial Sequence
  Synthetic oligonucleotide
accagtgactca

SEQ ID NO: 18 - DNA, 12 bp, Artificial Sequence
  Synthetic oligonucleotide
gaataccaagtc

SEQ ID NO: 19 - DNA, 12 bp, Artificial Sequence
  Synthetic oligonucleotide
gtagatcgtgta

SEQ ID NO: 20 - DNA, 12 bp, Artificial Sequence
  Synthetic oligonucleotide
taacgtgtgtgc

SEQ ID NO: 21 - DNA, 12 bp, Artificial Sequence
  Synthetic oligonucleotide
cattatggcgtg

SEQ ID NO: 22 - DNA, 12 bp, Artificial Sequence
  Synthetic oligonucleotide
ccaatacgcctg

SEQ ID NO: 23 - DNA, 12 bp, Artificial Sequence
  Synthetic oligonucleotide
gatctgcgatcc

SEQ ID NO: 24 - DNA, 12 bp, Artificial Sequence
  Synthetic oligonucleotide
cagctcatcagc

SEQ ID NO: 25 - DNA, 12 bp, Artificial Sequence
  Synthetic oligonucleotide
caaacaacagct

SEQ ID NO: 26 - DNA, 12 bp, Artificial Sequence
  Synthetic oligonucleotide
gcaacaccatcc

SEQ ID NO: 27 - DNA, 12 bp, Artificial Sequence
  Synthetic oligonucleotide
gcgatatatcgc

SEQ ID NO: 28 - DNA, 12 bp, Artificial Sequence
  Synthetic oligonucleotide
cgagcaatccta

SEQ ID NO: 29 - DNA, 12 bp, Artificial Sequence
  Synthetic oligonucleotide
agtcgtgcacat

SEQ ID NO: 30 - DNA, 12 bp, Artificial Sequence
  Synthetic oligonucleotide
gtatctgcgcgt

SEQ ID NO: 31 - DNA, 12 bp, Artificial Sequence
  Synthetic oligonucleotide
cgagggaaagtc

SEQ ID NO: 32 - DNA, 12 bp, Artificial Sequence
  Synthetic oligonucleotide
caaattcgggat

SEQ ID NO: 33 - DNA, 12 bp, Artificial Sequence
  Synthetic oligonucleotide
agattgaccaac

SEQ ID NO: 34 - DNA, 12 bp, Artificial Sequence
  Synthetic oligonucleotide
agttacgagcta

SEQ ID NO: 35 - DNA, 12 bp, Artificial Sequence
  Synthetic oligonucleotide
gcatatgcactg

SEQ ID NO: 36 - DNA, 12 bp, Artificial Sequence
  Synthetic oligonucleotide
caactcccgtga

SEQ ID NO: 37 - DNA, 12 bp, Artificial Sequence
  Synthetic oligonucleotide
ttgcgttagcag

SEQ ID NO: 38 - DNA, 12 bp, Artificial Sequence
  Synthetic oligonucleotide
tacgagccctaa

SEQ ID NO: 39 - DNA, 12 bp, Artificial Sequence
  Synthetic oligonucleotide
cactacgctaga

SEQ ID NO: 40 - DNA, 12 bp, Artificial Sequence
  Synthetic oligonucleotide
tgcagtcctcga

SEQ ID NO: 41 - DNA, 12 bp, Artificial Sequence
  Synthetic oligonucleotide
accatagctccg

SEQ ID NO: 42 - DNA, 12 bp, Artificial Sequence
  Synthetic oligonucleotide
tcgacatctctt

SEQ ID NO: 43 - DNA, 12 bp, Artificial Sequence
  Synthetic oligonucleotide
gaacactttgga

SEQ ID NO: 44 - DNA, 12 bp, Artificial Sequence
  Synthetic oligonucleotide
gagccatctgta

SEQ ID NO: 45 - DNA, 12 bp, Artificial Sequence
  Synthetic oligonucleotide
ttgggtacacgt

SEQ ID NO: 46 - DNA, 12 bp, Artificial Sequence
  Synthetic oligonucleotide
aaggcgctcctt

SEQ ID NO: 47 - DNA, 12 bp, Artificial Sequence
  Synthetic oligonucleotide
taatacggatcg

SEQ ID NO: 48 - DNA, 12 bp, Artificial Sequence
  Synthetic oligonucleotide
tcggaattagac

SEQ ID NO: 49 - DNA, 12 bp, Artificial Sequence
  Synthetic oligonucleotide
tgtgaattcgga

SEQ ID NO: 50 - DNA, 12 bp, Artificial Sequence
  Synthetic oligonucleotide
cattcgtggcgt

SEQ ID NO: 51 - DNA, 12 bp, Artificial Sequence
  Synthetic oligonucleotide
tactacgtggcc

SEQ ID NO: 52 - DNA, 12 bp, Artificial Sequence
  Synthetic oligonucleotide
ggccagttccta

SEQ ID NO: 53 - DNA, 12 bp, Artificial Sequence
  Synthetic oligonucleotide
gatgttcgctag

SEQ ID NO: 54 - DNA, 12 bp, Artificial Sequence
  Synthetic oligonucleotide
ctatctcctgtc

SEQ ID NO: 55 - DNA, 12 bp, Artificial Sequence
  Synthetic oligonucleotide
actcacaggaat

SEQ ID NO: 56 - DNA, 12 bp, Artificial Sequence
  Synthetic oligonucleotide
atgatgagcctc

SEQ ID NO: 57 - DNA, 12 bp, Artificial Sequence
  Synthetic oligonucleotide
gtcgacagagga

SEQ ID NO: 58 - DNA, 12 bp, Artificial Sequence
  Synthetic oligonucleotide
tgtcgcaaatag

SEQ ID NO: 59 - DNA, 12 bp, Artificial Sequence
  Synthetic oligonucleotide
catccctctact

SEQ ID NO: 60 - DNA, 12 bp, Artificial Sequence
  Synthetic oligonucleotide
tataccgctgcg

SEQ ID NO: 61 - DNA, 12 bp, Artificial Sequence
  Synthetic oligonucleotide
agttgaggcatt

SEQ ID NO: 62 - DNA, 12 bp, Artificial Sequence
  Synthetic oligonucleotide
acaatagacacc

SEQ ID NO: 63 - DNA, 12 bp, Artificial Sequence
  Synthetic oligonucleotide
cggtcaattgac

SEQ ID NO: 64 - DNA, 12 bp, Artificial Sequence
  Synthetic oligonucleotide
gtggagtctcat

SEQ ID NO: 65 - DNA, 12 bp, Artificial Sequence
  Synthetic oligonucleotide
gctcgaagattc

SEQ ID NO: 66 - DNA, 12 bp, Artificial Sequence
  Synthetic oligonucleotide
aggcttacgtgt

SEQ ID NO: 67 - DNA, 12 bp, Artificial Sequence
  Synthetic oligonucleotide
tctctaccactc

SEQ ID NO: 68 - DNA, 12 bp, Artificial Sequence
  Synthetic oligonucleotide
acttccaacttc

SEQ ID NO: 69 - DNA, 12 bp, Artificial Sequence
  Synthetic oligonucleotide
ctcacctaggaa

SEQ ID NO: 70 - DNA, 12 bp, Artificial Sequence
  Synthetic oligonucleotide
gtgttgtcgtgc

SEQ ID NO: 71 - DNA, 12 bp, Artificial Sequence
  Synthetic oligonucleotide
ccacagatcgat

SEQ ID NO: 72 - DNA, 12 bp, Artificial Sequence
  Synthetic oligonucleotide
tatcgacacaag

SEQ ID NO: 73 - DNA, 12 bp, Artificial Sequence
  Synthetic oligonucleotide
gattccggctca

SEQ ID NO: 74 - DNA, 12 bp, Artificial Sequence
  Synthetic oligonucleotide
cgtaattgccgc

SEQ ID NO: 75 - DNA, 12 bp, Artificial Sequence
  Synthetic oligonucleotide
ggtgactagttc

SEQ ID NO: 76 - DNA, 12 bp, Artificial Sequence
  Synthetic oligonucleotide
atgggttccgtc

SEQ ID NO: 77 - DNA, 12 bp, Artificial Sequence
  Synthetic oligonucleotide
taggcatgcttg

SEQ ID NO: 78 - DNA, 12 bp, Artificial Sequence
  Synthetic oligonucleotide
aactagttcagg

SEQ ID NO: 79 - DNA, 12 bp, Artificial Sequence
  Synthetic oligonucleotide
attctgccgaag

SEQ ID NO: 80 - DNA, 12 bp, Artificial Sequence
  Synthetic oligonucleotide
agcatgtcccgt

SEQ ID NO: 81 - DNA, 12 bp, Artificial Sequence
  Synthetic oligonucleotide
gtacgatatgac

SEQ ID NO: 82 - DNA, 12 bp, Artificial Sequence
  Synthetic oligonucleotide
gtggtggtttcc

SEQ ID NO: 83 - DNA, 12 bp, Artificial Sequence
  Synthetic oligonucleotide
tagtatgcgcaa

SEQ ID NO: 84 - DNA, 12 bp, Artificial Sequence
  Synthetic oligonucleotide
tgcgctgaatgt

SEQ ID NO: 85 - DNA, 12 bp, Artificial Sequence
  Synthetic oligonucleotide
atggctgtcagt

SEQ ID NO: 86 - DNA, 12 bp, Artificial Sequence
  Synthetic oligonucleotide
gttctcttctcg

SEQ ID NO: 87 - DNA, 12 bp, Artificial Sequence
  Synthetic oligonucleotide
cgtaagatgcct

SEQ ID NO: 88 - DNA, 12 bp, Artificial Sequence
  Synthetic oligonucleotide
gcgttctagctg

SEQ ID NO: 89 - DNA, 12 bp, Artificial Sequence
  Synthetic oligonucleotide
gttgttctggga

SEQ ID NO: 90 - DNA, 12 bp, Artificial Sequence
  Synthetic oligonucleotide
ggacttccagct

SEQ ID NO: 91 - DNA, 12 bp, Artificial Sequence
  Synthetic oligonucleotide
ctcacaaccgtg

SEQ ID NO: 92 - DNA, 12 bp, Artificial Sequence
  Synthetic oligonucleotide
ctgctattcctc

SEQ ID NO: 93 - DNA, 12 bp, Artificial Sequence
  Synthetic oligonucleotide
atgtcaccgctg

SEQ ID NO: 94 - DNA, 12 bp, Artificial Sequence
  Synthetic oligonucleotide
tgtaacgccgat

SEQ ID NO: 95 - DNA, 12 bp, Artificial Sequence
  Synthetic oligonucleotide
agcagaacatct

SEQ ID NO: 96 - DNA, 12 bp, Artificial Sequence
  Synthetic oligonucleotide
tggagtaggtgg

SEQ ID NO: 97 - DNA, 12 bp, Artificial Sequence
  Synthetic oligonucleotide
ttggctctattc

SEQ ID NO: 98 - DNA, 12 bp, Artificial Sequence
  Synthetic oligonucleotide
gatcccacgtac

SEQ ID NO: 99 - DNA, 21 bp, Artificial Sequence
  PCR primer
aatgatacggcgaccaccgag

SEQ ID NO: 100 - DNA, 23 bp, Artificial Sequence
  PCR primer
gtgactggagttcagacgtgtgc

SEQ ID NO: 101 - DNA, 57 bp, Artificial Sequence
  PCR primer
  misc_feature (25)..(34): n is a, c, g, or t
caagcagaagacggcatacgagatnnnnnnnnnngtgactggagttcagacgtgtgc